(19) United States
(12) Patent Application Publication
(10) Pub. No.: US 2006/0276910 A1
(43) Pub. Date: Dec. 7, 2006

(54) Endoprostheses

(76) Inventor: Jan Weber, Maple Grove, MN (US)

Correspondence Address: FISH & RICHARDSON P.C., PO BOX 1022, MINNEAPOLIS, MN, 55440-1022, US

(21) Appl. No.: 11/142,800

(22) Filed: Jun. 1, 2005

(51) Int. Cl. A61F 2/04; A61F 2/06

(52) U.S. Cl. 623/23.71; 623/1.15; 600/431

(57) ABSTRACT

Medical devices, such as endoprostheses, are disclosed. In some embodiments, an endoprosthesis includes a tubular body including a first material having a first mass attenuation coefficient; and a coating on less than or equal to half of a (e.g., any) circumferential cross section occupied by the body. The coating includes a second material having a second mass attenuation coefficient greater than the first mass attenuation coefficient. When placed in a body, the endoprosthesis can be imaged using multiple types of methods, such as computed tomography.

### TECHNICAL FIELD

The invention relates to medical devices, such as endoprostheses (e.g., stents).

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced, or even replaced, with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

When the endoprosthesis is advanced through the body, its progress can be monitored, e.g., tracked, so that the endoprosthesis can be delivered properly to a target site. After the endoprosthesis is delivered to the target site, the endoprosthesis can be monitored to determine whether it has been placed properly and/or is functioning properly. The lumen in which the endoprosthesis is placed can also be monitored to determine whether it has renarrowed. Methods of monitoring include X-ray fluoroscopy, magnetic resonance imaging (MRI), and computed tomography (CT).

In computed tomography, a CT scanner is used to construct two- and three-dimensional images from multiple scans. The CT scanner has an X-ray source mounted on a circular track, and an arc-shaped detector also mounted on the track and opposite to the X-ray source. During use, the patient is positioned such that the track surrounds the patient. The X-ray source and the detector are then moved along the track, while the X-ray source emits an X-ray beam at multiple angles, and the detector detects the X-rays transmitted through the patient and the endoprosthesis. The X-rays detected by the detector are then sent to a computer for processing and forming the desired two- and three-dimensional images for display.

### SUMMARY

The invention relates to medical devices, such as endoprostheses.

In one aspect, the invention features an endoprosthesis having a tubular body including a first material and a second material. The first material has a first mass attenuation coefficient and the second material has a second mass attenuation coefficient greater than the first mass attenuation coefficient. The second material is on greater than zero to 50% of a circumferential cross section defined by the body.

Embodiments may include one or more of the following features. The second material can be on greater than zero to forty percent of any circumferential cross section defined by the body. The body can have a pattern of cells defined by bands, where at least one of the cells comprises one or more bands surrounding an aperture and at least one of the cells comprises one or more bands surrounding a solid area and forms a solid cell including the first material; the second material can contact at least a portion of the solid cell. The second material can be on less than or equal to about twenty percent of any circumferential cross section defined by the body. The second material can be on less than or equal to about one eighth of any circumferential cross section defined by the body. The second material can be substantially non-biodegradable. The second material can be located at one or both ends of the body. A cross-sectional portion between the ends of the body can be free of the second material. The second material can be located along a length of the body. The second material can be located at a series of discontinuous portions along a length of the body. The second material can extend spirally along the body. At least a portion of the second material can be at least about five microns thick. The second material can have a density greater than about 9.9 g/cm³. The second material can be formed as two separate portions, each portion on opposing circumferential areas of the body. The second material can be selected from the group consisting of tantalum, titanium, zirconium, iridium, palladium, hafnium, tungsten, gold, ruthenium, rhenium, barium, dysprosium, gadolinium and platinum. The second material can include an alloy. The endoprosthesis can include a drug. The second material can be disposed outwardly relative to the body. A biodegradable coating can be on the body, the biodegradable coating comprising a third material having a third mass attenuation coefficient higher than the first mass attenuation coefficient.

In yet another aspect, the invention features a method including obtaining an image of an endoprosthesis in a body using computed tomography, the endoprosthesis comprising a tubular body including a first material having a first mass attenuation coefficient, and a second material on less than or equal to half of a circumferential cross section defined by the body, the second material having a second mass attenuation coefficient greater than the first mass attenuation coefficient.

Embodiments of the method may include one or more of the following features. Obtaining the image can include determining a first and a second set of images from a plurality of computed tomography scan images, wherein the first set of images display a higher percentage of the second material than the second set of images. The method can include forming a final image from the second set of images. The determining step can determine a set of images that display less than a predetermined amount of the second material.

In yet another aspect, the invention features a method including obtaining a plurality of computed tomography scan images of a body having the endoprosthesis located therein. Images that display the endoprosthesis are determined from the plurality of computed tomography scan images. Selected images that display the endoprosthesis are subtracted from the plurality of computed tomography scans to determine a set of desired images. The selected images can display a higher percentage of the coating than a second set of images. A final image is formed from the desired images.

In another aspect, the invention features an implantable filter having a plurality of elongated members having a first material with a first mass attenuation coefficient, at least one elongated member having a second material with a second mass attenuation coefficient higher than the first mass attenuation coefficient, and at least one elongated member being free of the second material.

Embodiments may include one or more of the following advantages. A stent partially coated with radiopaque material allows a physician the freedom to use a wider range of imaging techniques for observation and diagnosis. Both fluoroscopic imaging and CT imaging can be useful to the physician for different purposes and at different times of treating or monitoring a patient. A stent that is viewable using either imaging techniques provides greater flexibility to a physician wanting to monitor the patient's health or to diagnose disease. In comparison, certain stents may not be fully compatible with CT imaging, because the X-ray attenuation or radiopacity of materials used in the stents may be too high for CT imaging. For example, images of stents fully coated with radiopaque material obtained by CT angiography can produce blooming artifacts and artificial thickening of the stent components that are displayed. These effects can lead to image artifacts that interfere with lumen visualization and quantification.

Other aspects, features, and advantages will be apparent from the description of the preferred embodiments thereof and from the claims.

### DESCRIPTION OF DRAWINGS

FIG. 1 is a perspective view of an embodiment of an expanded stent; FIG. 2A is a cross section of the stent of FIG. 1, taken along line 2A-2A; and FIG. 2B is a cross section of the stent of FIG. 1, taken along line 2B-2B.

FIG. 3 is a diagrammatic view of a stent during a computed tomography procedure.

FIG. 4 is cross section of a stent with two coating portions.

FIG. 5 is a diagrammatic view of a stent with two coating portions during a computed tomography procedure.

FIGS. 6, 7 and 8 are perspective views of embodiments of expanded stents.

FIGS. 9 and 10 are side views of embodiments of expanded stents.

FIG. 11 is a flow chart of an embodiment of a method of forming a stent.

FIG. 12 is a flow chart of an embodiment of a method of imaging a stent.

FIG. 13 is a schematic of fluoroscopic imaging of a body with a stent embedded therein.

FIG. 14 is a schematic of computed tomography imaging of a body with a stent embedded therein.

FIG. 15 is a perspective view of an embodiment of a stent.

FIG. 16 is a cross section of an embodiment of a stent.

### DETAILED DESCRIPTION

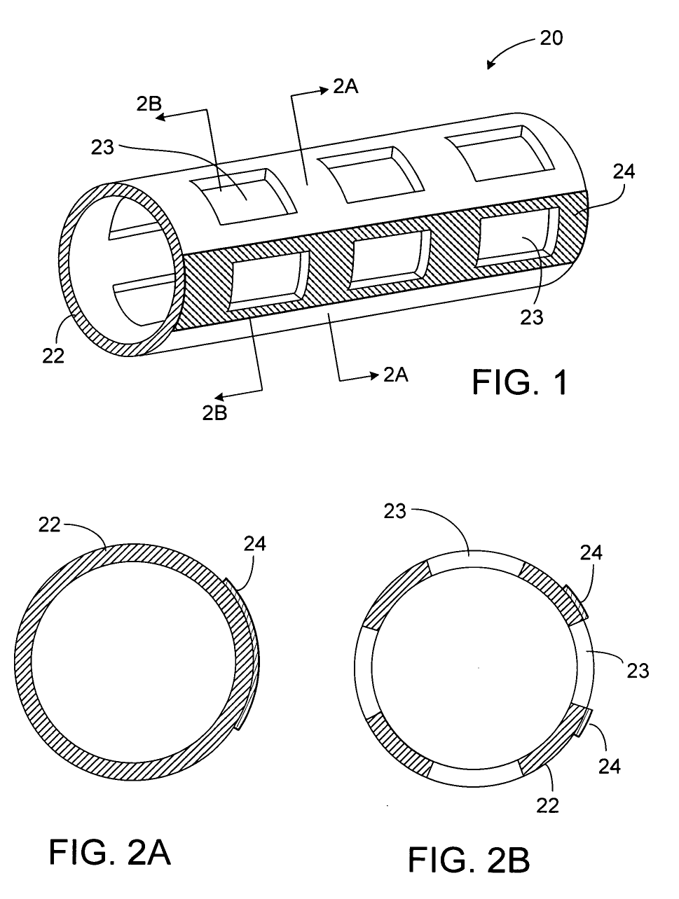

Referring to FIGS. 1, 2A and 2B, a stent 20 includes a tubular body 22 having a plurality of openings 23, and a coating 24 on a portion of the tubular body. Tubular body 22 can be made of a biocompatible material with mechanical properties that allow stent 20 to be compacted and subsequently expanded to support a vessel, such as stainless steel, magnesium alloy or a nickel-titanium alloy. Coating 24 can be made of a radiopaque material, such as platinum or gold. Along one or more circumferential cross sections of stent 20, coating 24 covers less than or equal to 50% of the circumference occupied by tubular body 22. For example, as shown in FIG. 2A, coating 24 covers less than 25% of the circumference occupied by tubular body 22.

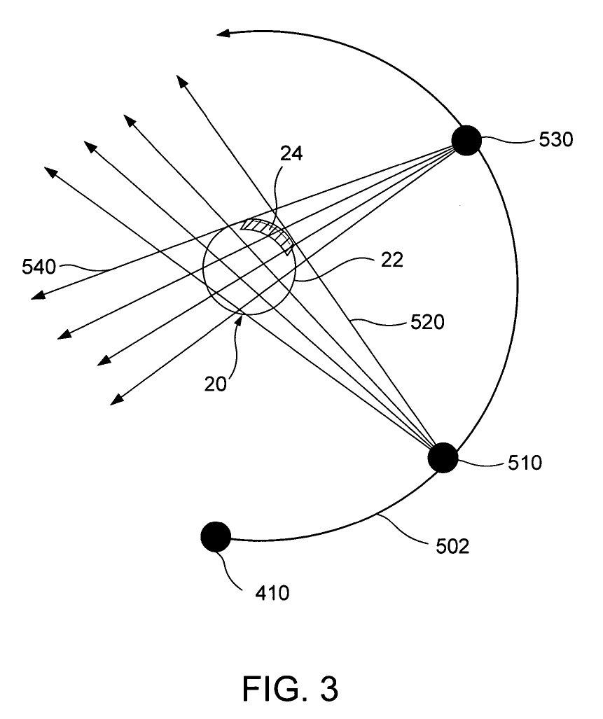

Coating 24 is capable of enhancing the visibility of stent 20 under X-ray visualization techniques, such as fluoroscopy, and particularly under computed tomography (CT). Referring to FIG. 3, stent 20 is shown in a CT scanner having an X-ray source 410 mounted on a circular track 502. During a computed tomography procedure, X-ray source 410 moves along track 502 and emits X-rays 520, 540 while a detector (not shown) mounted on the track opposite the X-ray source 410 detects X-rays transmitted through the implanted stent 20. Scans from different angles are taken along track 502 to generate the desired images to be displayed. As shown in FIG. 3, at point 510, the cross section of the stent that is intersected by X-rays 520 and that is relatively radiopaque is small, and most of the X-rays 520 pass through the relatively radiolucent tubular body 22 of the stent. That is, at point 510, X-rays 520 produce an image with relatively little of radiopaque coating 24. In comparison, at point 530, many of the X-rays 540 impinge upon radiopaque coating 24 to produce an image with a higher amount of the radiopaque coating 24. The images produced from point 530 indeed can be too highly visible (e.g., bright) and obscure visualization of the stent 20, the vessel in which the stent 20 is placed, and the surrounding tissue. But by collecting the desired images from different points along track 502, eliminating those images that are too radiopaque (e.g., at point 530), and keeping images that are less radiopaque, more useful images can be constructed and displayed. In comparison, stents that are fully coated with radiopaque material do not offer the option of eliminating CT images that are too highly visible because the levels of X-ray attenuation are relatively uniform about the circumference of the stent. During a CT procedure, the fully coated stents may show blooming artifacts or artificial thickening of the stent structure that impede visualization and quantification of the vessel lumen.

Referring again to FIG. 1, tubular body 22 can include (e.g., be manufactured from) one or more biocompatible materials with mechanical properties so that stent 20 can be compacted, and subsequently expanded. In some embodiments, stent 20 can have an ultimate tensile strength (UTS) of about 20-150 kPSI, greater than about 15% elongation to failure, and a modulus of elasticity of about 10-60 MPSI. When stent 20 is expanded, the material can be stretched to strains on the order of about 0.3. Examples of “structural” materials that provide good mechanical properties (e.g., sufficient to support a lumen wall) and/or biocompatibility include, for example, stainless steel (e.g., 316L and 304L stainless steel, and PERSS®), Nitinol (a nickel-titanium alloy), Elgiloy, L605 alloys, MP35N, Ti-6Al-4V, Ti-50Ta, Ti-10Ir, Nb-1Zr, Ti-4Al-4Mo-4Sn-0.5Si (551) and Co-28Cr-6Mo. Because of its low radiopacity, a magnesium alloy with a corrosion resistant surface treatment or a corrosion resistant magnesium alloy can also be used. Other materials include elastic biocompatible metal such as a superelastic or pseudo-elastic metal alloy, as described, for example, in Schetsky, L. McDonald, “Shape Memory Alloys”, Encyclopedia of Chemical Technology (3rd ed.), John Wiley & Sons, 1982, vol. 20. pp. 726-736; and commonly assigned, Stinson, US 2004/0143317 A1. Tubular body 22 can include (e.g., be formed of) a biodegradable metal or a polymer (e.g., a biodegradable polymer), as described in Bolz, U.S. Pat. No. 6,287,332; Heublein, US 2002/0004060 A1; U.S. Pat. No. 5,587,507; and U.S. Pat. No. 6,475,477. Tubular body 22 can include two or more layers, for example of different compositions. In some embodiments, the material(s) of tubular body 22 is less radiopaque or more radiolucent than the material(s) of coating 24.

Coating 24 can be made of one or more biocompatible materials capable of enhancing the radiopacity of body 22, for example, by having a higher density or mass attenuation coefficient. Examples of radiopaque materials include metallic elements having atomic numbers greater than 26, e.g., greater than 43. In some embodiments, the radiopaque materials have a density greater than about 9.9 g/cc. In certain embodiments, the radiopaque material is relatively absorptive of X-rays, e.g., having a linear attenuation coefficient of at least 25 cm⁻¹, e.g., at least 50 cm⁻¹, at 100 keV. Some radiopaque materials include tantalum, platinum, iridium, palladium, hafnium, zirconium, tungsten, molybdenum, gold, ruthenium, bismuth, and rhenium. Oxides of radiopaque materials, such as bismuth oxide and zirconium oxide, can be used. The radiopaque material can include an alloy, such as a binary, a ternary or more complex alloy, containing one or more elements listed above with one or more other elements such as iron, nickel, cobalt, or titanium. Examples of alloys including one or more radiopaque materials are described in U.S. Application Publication US-2003-0018380-A1; US-2002-0144757-A1; and US-2003-0077200-A1. Combinations of any of the above materials can also be used.

In some embodiments, coating 24 includes one or more organic components and one or more of the radiopaque materials described above. The organic component(s) can include a biocompatible polymer that is biodegradable or non-biodegradable. Examples of polymers include polytetrafluoroethylene (PTFE), expanded PTFE, polyethylene, urethane, or polypropylene. Examples of biodegradable polymers are described in U.S. Pat. No. 5,587,507; and U.S. Pat. No. 6,475,477.

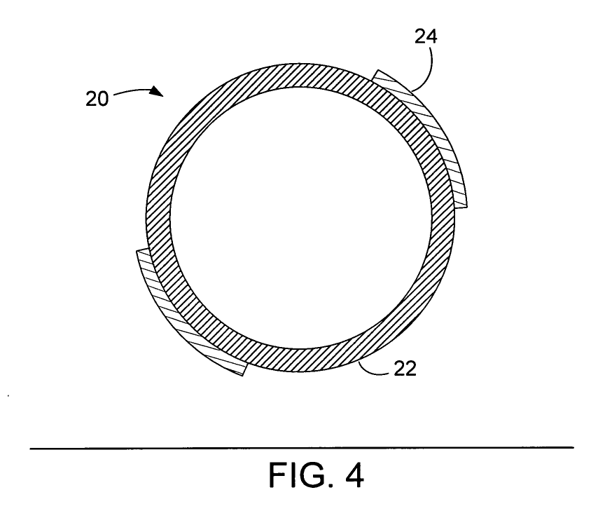

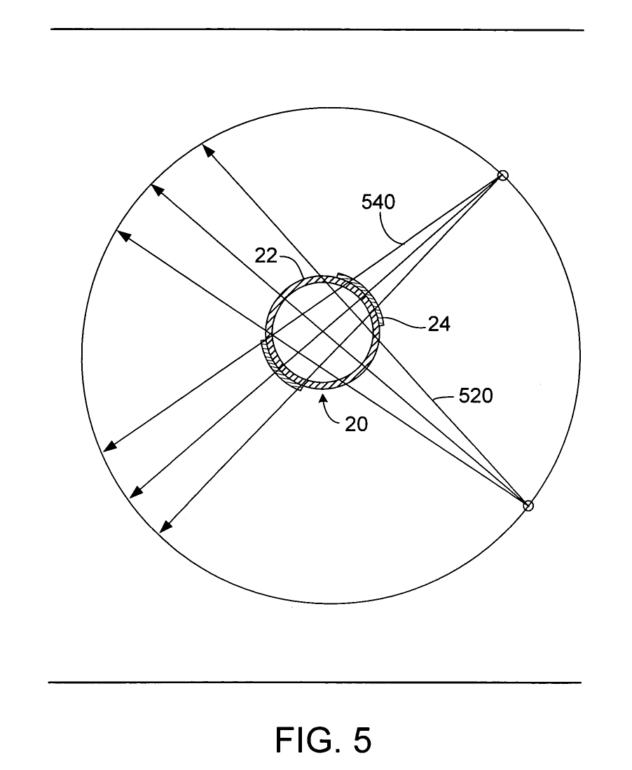

Referring to FIG. 4, in some implementations, the coating 24 is applied to two portions of the stent, where the two portions are substantially opposite along the circumference of the stent. As shown in FIG. 5, the X-rays 540 passing through the radiopaque coating 24 of the stent pass through both coatings when the coatings are opposite to one another.

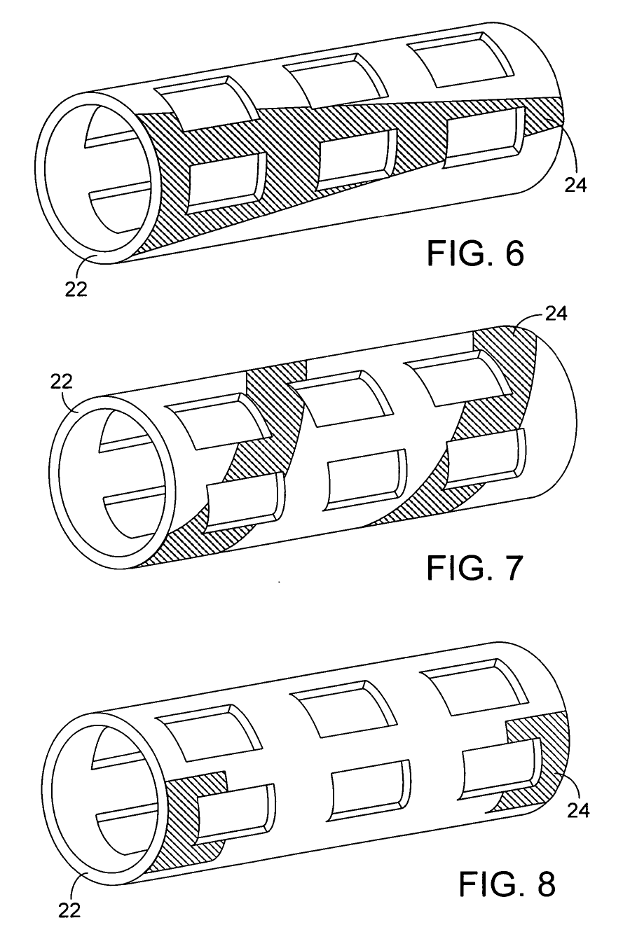

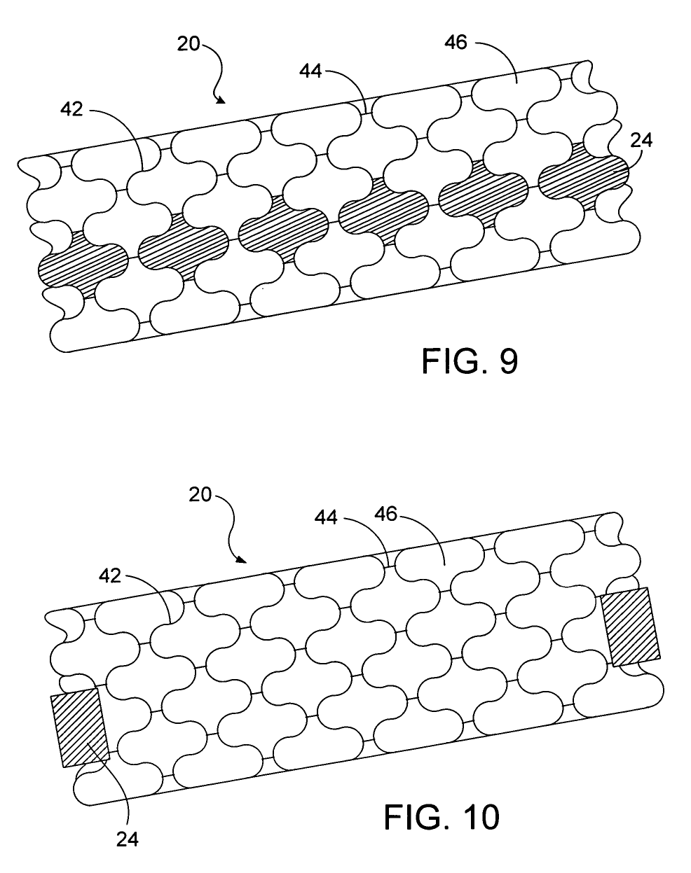

As indicated above, coating 24 covers less than or equal to 50%, such as less than about 20%, of a circumference occupied by tubular body 22. The circumference occupied by tubular body 22 can be equal to or less than the circumference generally defined by the tubular body. For example, in the cross section shown in FIG. 2A, the circumference occupied by tubular body 22 is equal to the circumference defined by the tubular body, which is measured along the exterior surface of the tubular body. But at the cross section shown in FIG. 2B, which intersects openings 23, the circumference occupied by the tubular body is equal to the circumference defined by the tubular body at that cross section, minus the circumference defined by the openings. Other embodiments of stents in which the circumference occupied by the tubular body is less than the circumference defined by the tubular body include stents formed by knitting or weaving wires, and stents having bands connected by connectors (as shown below in FIGS. 9 and 10). Coating 24 can cover greater than or equal to zero percent, about 5%, about 10%, about 15%, about 20%, about 25%, about 30%, about 35%, about 40%, or about 45% of a circumference occupied by tubular body 22; and/or less than or equal to 50%, about 45%, about 40%, about 35%, about 30%, about 25%, about 20%, about 15%, about 10%, or about 5% of a circumference defined by the tubular body. The degree to which coating 24 extends along a circumference of a stent can vary or be constant along the length of the stent (FIG. 6).

The thickness of coating 24 can also vary, and can be dependent, for example, on the type of stent, the material and or/ thickness from which the body 22 is formed, the degree to which the coating covers the stent, and the composition of the coating. In some embodiments, the thickness of coating 24 is at least about five microns thick. In one embodiment, a stent that is about 80 microns thick and formed of magnesium having a partial coating of gold that is at least about 8 microns thick is sufficiently visible to under fluoroscopy. The thickness can be determined by the mass attenuation coefficient of the material used to form the coating. As an example of the coating thickness, the coating 24 (or stent 20 with the coating 24) can be formed to be sufficiently thick to be as radiopaque as a stainless steel stent having a strut thickness of about 80 microns, which is sufficient radiopaque to 80 keV fluoroscopy X-rays. The mass attenuation coefficient of the coating 24 plus any material under the coating, such as the tubular body 22, can be used to determine how thick the coating needs be for the stent 20 to have radiopaque portions. Changing the materials, the X-ray voltage or thickness of the body 22 can change the required thickness of the coating 24. Coating compositions having high density materials or high atomic numbers may be thinner than materials having low density or low atomic numbers. Stents with high coating coverage may be thinner than low coating coverage. The thickness of coating 24 can vary along a stent.

Coating 24 can be formed anywhere along an axial direction of stent 20. For example, coating 24 can be on the exterior surface of stent 20 and/or on the interior surface of the stent. In embodiments in which tubular body 22 includes multiple layers, coating 24 can be between two or more layers of the tubular body. More than one coating can be formed along an axial direction. For example, along an axial direction, a stent may include a radiopaque coating on the exterior surface and one or more coatings between the exterior surface and the interior surface.

The manner in which coating 24 extends along stent 20 can also vary. For example, as shown in FIG. 1, coating 24 can extend generally linearly and uninterruptedly from one end of the stent to the other end. In other embodiments, referring to FIG. 7, coating 24 extends non-linearly, as shown, spirally, about the stent. Coating 24 can also extend discontinuously along the length of the stent such that two or more areas of coating 24 are separated by one or more portions of uncoated stent. For example, FIG. 8 shows stent 20 with both ends having coating 24 of radiopaque material. Coating stent 20 at one or both ends can enable the ends of stent 20 to be detected. If determining the position of the end of stent 20 is desired, such as when multiple stents are aligned in a row, coating the ends can increase the visibility of the ends of stent 20. Coating 24 can extend along less than the entire length of a stent. For example, coating 24 can be located only at end portions (as shown in FIG. 8) or the coating can be located only one or more portions between the end portions.

Still other embodiments of coated stents can be formed. FIG. 9 shows stent 20 in the form of a tubular member defined by a plurality of bands 42 and connectors 44 that extend between and connect adjacent bands. Bands 42 and connectors 44 define the perimeter of a cell 46. Each cell 46 can be an open cell, that is, bands 22 and connectors 24 surround an aperture; or each cell 46 can be a closed cell, for example, the cell can have a solid surface made of a stent material. In some embodiments, most of the cells 46 are open cells. To the closed cells, coating 24 can be applied. As shown in FIG. 9, cells having a coating 24 can be adjacent to one another. Alternatively, one or more non-coated cells can be between cells having coating 24. When cells 46 are coated, a whole cell can be coated with radiopaque material, or only a portion of cell 46 can be coated. Referring to FIG. 10, coating 24 can be applied such that the coating does not completely correspond to one or more cells, but covers a portion of stent cells.

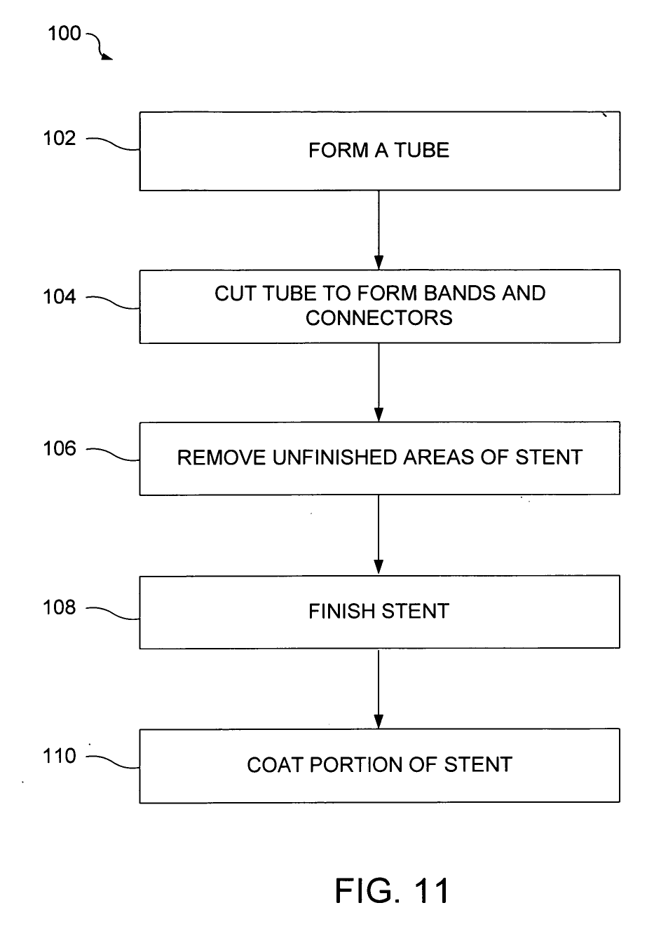

FIG. 11 shows a method 100 of making stent 20. As shown, method 100 includes forming a tube (step 102) that makes up tubular body 22 of stent 20. The tube is subsequently cut to form openings (or bands 22 and connectors 24) (step 104) to produce an unfinished stent. Areas of the unfinished stent affected by the cutting are subsequently removed (step 106). The unfinished stent is finished (step 108). One or more portions of stent 20 is coated with a radiopaque material (step 110), and the stent can then be further finished.

The tube that makes up the tubular member of stent 20 can be formed using metallurgical techniques, such as thermomechanical processes (step 102). For example, a hollow metallic member (e.g., a rod or a bar) can be drawn through a series of dies with progressively smaller circular openings to plastically deform the member to a targeted size and shape. In some embodiments, the plastic deformation strain hardens the member (and increases its yield strength) and elongates the grains along the longitudinal axis of the member. The deformed member can be heat treated (e.g., annealed above the recrystallization temperature and/or hot isostatically pressed) to transform the elongated grain structure into an initial grain structure, e.g., one including equiaxed grains. Small or fine grains can be formed by heating the member close to the recrystallization temperature for a short time. Large or coarse grains can be formed by heating the member at higher temperatures and/or for longer times to promote grain growth.

Next, openings (or bands 22 and connectors 24) of stent 20 are formed, as shown, by cutting the tube (step 104). Selected portions of the tube can be removed to form bands 22 and connectors 24 by laser cutting, as described in U.S. Pat. No. 5,780,807, hereby incorporated by reference in its entirety. In certain embodiments, during laser cutting, a liquid carrier, such as a solvent or an oil, is flowed through the lumen of the tube. The carrier can prevent dross formed on one portion of the tube from re-depositing on another portion, and/or reduce formation of recast material on the tube. Other methods of removing portions of the tube can be used, such as mechanical machining (e.g., micro-machining), electrical discharge machining (EDM), and photoetching (e.g., acid photoetching).

In some embodiments, after bands 22 and connectors 24 are formed, areas of the tube affected by the cutting operation above can be removed (step 106). For example, laser machining of bands 22 and connectors 24 can leave a surface layer of melted and resolidified material and/or oxidized metal that can adversely affect the mechanical properties and performance of stent 20. The affected areas can be removed mechanically (such as by grit blasting or honing) and/or chemically (such as by etching or electropolishing).

The unfinished stent is then finished (step 108). The unfinished stent can be finished, for example, by chemical milling and/or electropolishing to a smooth finish.

Coating 24 of radiopaque material is then applied to one or more selected portions of the stent (step 110). The radiopaque material can be deposited, for example, using chemical vapor deposition, sputtering, physical vapor deposition, and/or laser pulse vapor deposition. A mandrel can be placed inside of the stent to prevent the radiopaque material from being applied to portions of the stent other than where the material is desired. A mask can be placed between the stent and the source of the radiopaque material to control the area of the stent to which the material is applied. Other coating methods can also be used, such as masking the portions of the stent which are not to be coated and dipping the stent in radiopaque material. A coating, such as a drug-eluting polymer coating, can be coated onto a portion of the stent and radiopaque particles can be mechanically pressed into the polymer coating. In one embodiment, the polymer can be made tacky so that the particles stick to the coating. Alternatively, radiopaque particles can be attached to stent 20 with an adhesive coating.

Stent 20 can be formed of a desired shape and size (e.g., coronary stents, aortic stents, peripheral vascular stents, gastrointestinal stents, urology stents, and neurology stents). Depending on the application, stent 20 can have a diameter of between, for example, 1 mm to 46 mm. In certain embodiments, a coronary stent can have an expanded diameter of from about 2 mm to about 6 mm. In some embodiments, a peripheral stent can have an expanded diameter of from about 5 mm to about 24 mm. In certain embodiments, a gastrointestinal and/or urology stent can have an expanded diameter of from about 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. Stent 20 can be balloon-expandable, self-expandable, or a combination of both (e.g., as described in U.S. Pat. No. 5,366,504).

In use, stent 20 can be used, e.g., delivered and expanded, using a catheter delivery system (step 202). Catheter systems are described in, for example, Wang U.S. Pat. No. 5,195,969, Hamlin U.S. Pat. No. 5,270,086, and Raeder-Devens, U.S. Pat. No. 6,726,712. Stents and stent delivery are also exemplified by the Radius® or Symbiot® systems, available from Boston Scientific Scimed, Maple Grove, Minn.

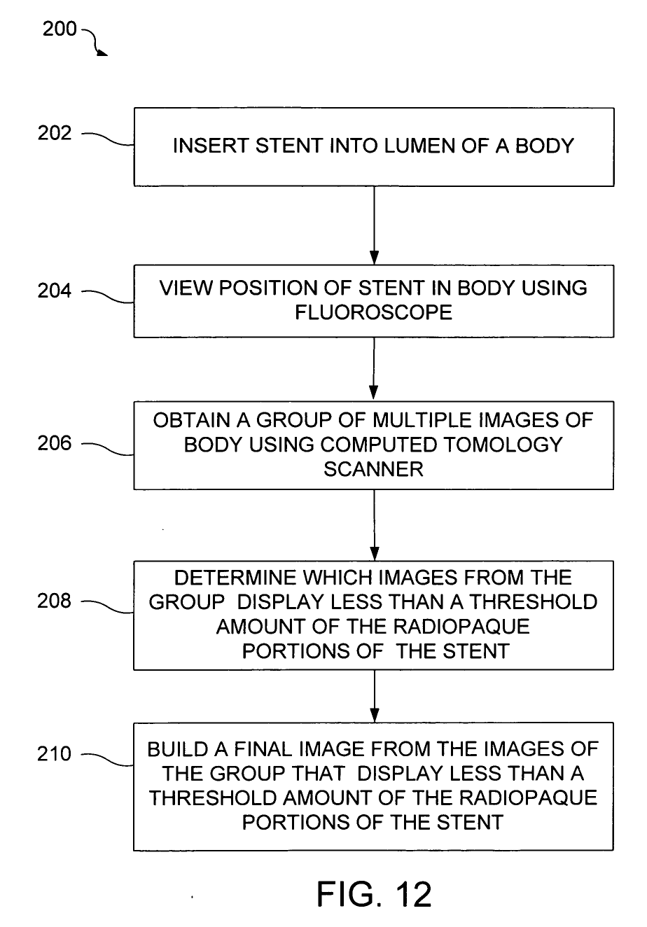

During and/or after stent delivery, stent 20 can be imaged using X-ray fluoroscopy and/or computed axial tomography. FIG. 12 shows an illustrative method 200 that includes using multiple methods to image stent 20 in a lumen. First, stent 20 is inserted into a body, such as into a lumen, for example, an artery (step 202). During delivery, X-ray fluoroscopy can be used to image stent 20 within the body by focusing X-rays on the body in the vicinity of the location of stent 20, detecting the X-rays that have passed through the body, and displaying an image on a monitor (step 204). Alternatively or additionally, stent 20 can be monitored in the body by capturing a group of images with a computed axial tomography (CAT or CT) device (step 206). Of the images that are captured by the CT scans, some of the images display a substantial amount of radiopaque coating 24, while other images display less than a threshold amount of the radiopaque coating (e.g., relatively little to virtually none of the radiopaque coating 24). The images that display less than a threshold amount of radiopaque coating 24 of stent 20 are determined (step 208). A final display image is built from the images that show less than a threshold amount of radiopaque coating 24 (step 210). In other embodiments, only one imaging technique, such as CT, is used during and after stent delivery.

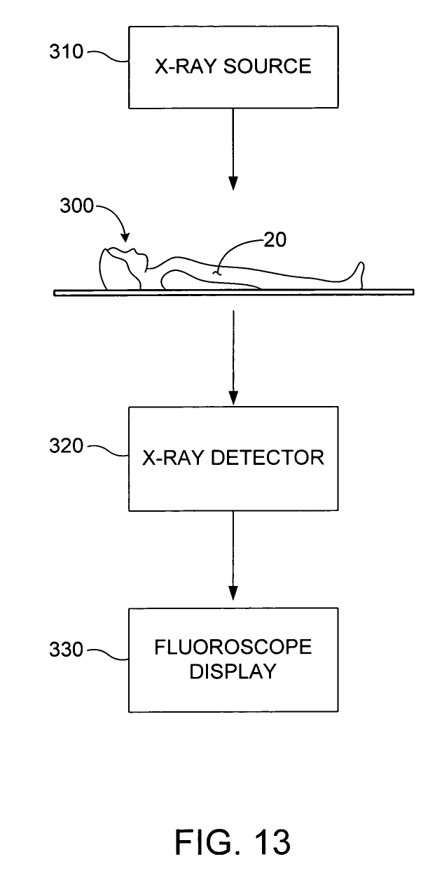

Referring also to FIG. 13, stent 20 can be viewed in the body using X-ray fluoroscopy (step 204). During fluoroscopy, an X-ray source 310 emits X-rays that are directed through body 300. An X-ray detector 320 detects the X-rays after the X-rays have passed through the body 300 and stent 20 to capture signals. The signals are then sent to a display 330, such as a monitor or computer screen, which displays a corresponding image.

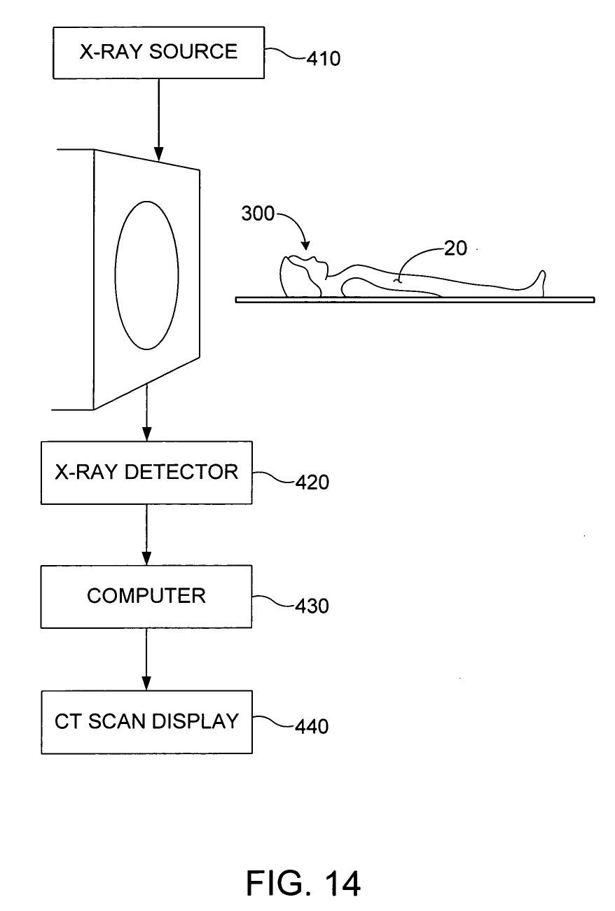

Referring to FIGS. 3 and 14, stent 20 can also be viewed in the body using a CT scanner (step 206). The CT scanner is used to construct two- and three-dimensional images from multiple images. The CT scanner has a rotating gantry with an X-ray source 410, such as an X-ray tube, mounted on one side and an arc-shaped detector mounted on the opposite side. The X-ray source moves along a circular track 502, starting at point 500 and moving toward point 510 and 530. The X-ray source emits an X-ray beam in a fan shape as the X-ray source and detector are rotated around body 300. At various points along the track 502, images are obtained. Approximately 1000 images may be obtained for each rotation of the X-ray source. Images are obtained up and down at least a portion of body 300. The images are obtained when the X-ray source 410 emits X-rays through body 300. An X-ray detector 420 detects the X-rays after they have passed through the body 300. The images are sent to a computer 430.

As the X-ray source 410 moves around body 300, images from different angles of body 300 and stent 20 are captured. At point 510, most of X-rays 520 pass through a portion of stent 20 that is includes tubular body 22, which is relatively radiolucent. At point 510, X-rays 520 emitted from X-ray source 410 produce relatively few images that show radiopaque coating 24. In comparison, at point 530, many of the X-rays impinge upon radiopaque coating 24 of stent 20 to produce images of the radiopaque coating. Of course, additional images can be captured at other points along track 502 and beyond, and FIG. 3 shows only points 510 and 530 for simplicity and clarity.

To improve the final image obtained by CT device, the initial images captured by the CT scanner can be examined to determine which of the images display more than a threshold amount of radiopaque coating 24 and which of the images display less than a threshold amount of the radiopaque coating (step 208). The images that display more than a threshold amount of radiopaque coating 24 may produce blooming artifacts and/or artificial thickening of the components of stent 20, and can be ignored in forming the image that is displayed. For example, the images captured at point 530 show much more of the radiopaque material than the images captured at point 510. Images obtained at points that display less than a threshold amount of radiopaque coating 24, such as at point 510, are selected for calculating the displayed image.

In some implementations, to determine the threshold amount of radiopaque coating 24, images are obtained at all points around the body. All the data points are used to determine the location of the stent in the body. Using the images that show the stent, images from a fraction of the circle are calculated. For example, if the stent is designed so that 50% of the images are usable, the data from a first portion of the images, such as the images obtained between 0 to 90°, can be calculated. Then, data from a second portion, for example, where the second portion is 10° offset from the first portion (images obtained between 10 to 100°), is calculated. The calculations are repeated until images from around 180° of the stent are calculated, because the other half of the stent is symmetric to the first half. The least absorbing set of images are then selected. The step size, described above as being 10°, can be fine tuned, such as to 5°. Thus, if the set of images between 40-130° is the best set of images, the calculation can be fine tuned between 35-125° and 45-135°.

From the images that display less than a threshold amount of radiopaque coating 24, a display image is formed (step 210). Building the final image can include compositing the individual images to obtain the final two- or three-dimensional image or images.

While a number of embodiments have been described above, the invention is not so limited.

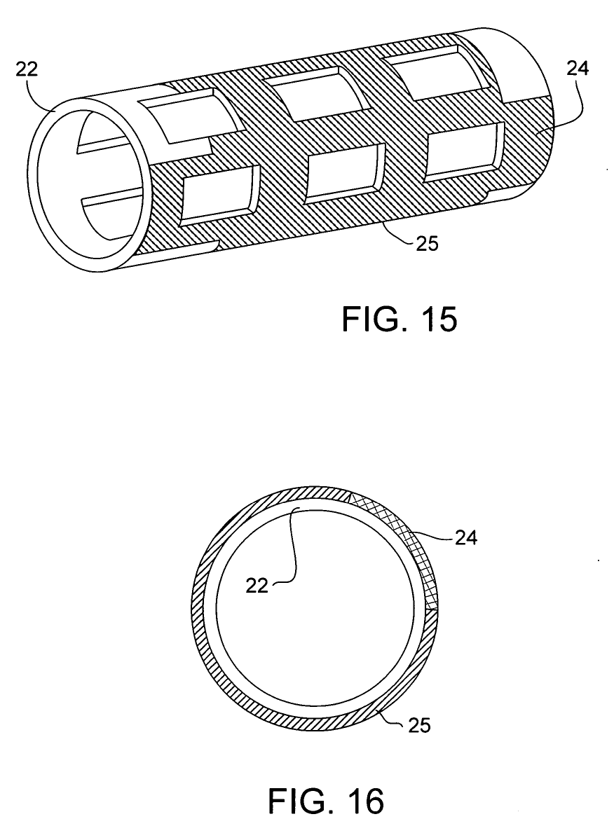

For example, referring to FIG. 15, a stent may include one or more portions 25 in which radiopaque coating 24 extends more than 50% of the circumference of the stent, for example, completely around the circumference. The portion(s) of coating 24 that extends more than 50% of the circumference of the stent can enhance visibility during fluoroscopy, while portion(s) of the coating that extends less than or equal to 50% of the circumference of the stent can enhance visibility during CT.

In some embodiments, stent 20 includes a releasable therapeutic agent, drug, or a pharmaceutically active compound. The agent, drug, or compound can be incorporated in radiopaque coating 24 (e.g., a polymeric radiopaque coating) and/or as a separate coating. Examples of releasable therapeutic agents, drugs, or a pharmaceutically active compounds are described in U.S. Pat. No. 5,674,242, Zhong, US 2003/003220 A1, and Lanphere US 2003/0185895 A1. The therapeutic agents, drugs, or pharmaceutically active compounds can include, for example, anti-thrombogenic agents, antioxidants, anti-inflammatory agents, anesthetic agents, anti-coagulants, and antibiotics.

Stent 20 can be a part of a covered stent or a stent-graft. In other embodiments, stent 20 can include and/or be attached to a biocompatible, non-porous or semi-porous polymer matrix made of polytetrafluoroethylene (PTFE), expanded PTFE, polyethylene, urethane, or polypropylene.

In some embodiments, in addition to coating 24, a stent includes a radiopaque, bioabsorbable coating. Referring to FIG. 16, stent 20 can include radiopaque coating 24 extending about a portion of the circumference of the stent, and a radiopaque, bioabsorbable coating 25 that extends about the remaining portion of the circumference of the stent. Coating 25 is capable of enhancing the radiopacity of stent 20, for example, under fluoroscopy during stent delivery. After the stent has been implanted, coating 25 can be bioabsorbed, thereby leaving coating 24 to enhance visibility during CT. Coating 25 can include a bioabsorbable polymer and a radiopaque material, as described above. In some embodiments, coating 25 only covers a portion of the circumference of the stent not covered by coating 24.

The radiopaque coatings described herein can be applied to other medical devices, such as filters. A filter can include a porous portion for filtering and a struts for supporting the porous portion. One or more of the struts can be fully or partially coated with radiopaque material.

In some embodiments, stent 20 includes one or more materials that enhance visibility by magnetic resonance imaging (MRI). Examples of MRI materials include non-ferrous metal-alloys containing paramagnetic elements (e.g., dysprosium or gadolinium) such as terbium-dysprosium, dysprosium, and gadolinium; non-ferrous metallic bands coated with an oxide or a carbide layer of dysprosium or gadolinium (e.g., Dy₂O₃ or Gd₂O₃); non-ferrous metals (e.g., copper, silver, platinum, or gold) coated with a layer of superparamagnetic material, such as nanocrystalline Fe₃O₄, CoFe₂O₄, MnFe₂O₄, or MgFe₂O₄; and nanocrystalline particles of the transition metal oxides (e.g., oxides of Fe, Co, Ni). Alternatively or in addition, stent 20 can include one or more materials having low magnetic susceptibility to reduce magnetic susceptibility artifacts, which during imaging can interfere with imaging of tissue, e.g., adjacent to and/or surrounding the stent. Low magnetic susceptibility materials include tantalum, platinum, titanium, niobium, copper, and alloys containing these elements. The MRI visible materials can be incorporated into the structural material, can serve as the structural material, and/or be included as one or more layers of stent 20.

All publications, references, applications, and patents referred to herein are incorporated by reference in their entirety.

Other embodiments are within the claims.

What is claimed is:

1. An endoprosthesis, comprising:
a tubular body including a first material having a first mass attenuation coefficient; and
a second material on the body, the second material being on greater than zero to 50% of a circumferential cross section defined by the body, the second material having a second mass attenuation coefficient greater than the first mass attenuation coefficient.

2. The endoprosthesis of claim 1, wherein the second material is on greater than zero to forty percent of any circumferential cross section defined by the body.

3. The endoprosthesis of claim 1, wherein:
the body has a pattern of cells defined by bands and at least one of the cells comprises one or more bands surrounding an aperture and at least one of the cells comprises one or more bands surrounding a solid area and forming a solid cell including the first material; and
the second material contacts at least a portion of the solid cell.

4. The endoprosthesis of claim 1, wherein the second material is on less than or equal to about twenty percent of any circumferential cross section defined by the body.

5. The endoprosthesis of claim 1, wherein the second material is on less than or equal to about one eighth of any circumferential cross section defined by the body.

6. The endoprosthesis of claim 1, wherein the second material is substantially non-biodegradable.

7. The endoprosthesis of claim 1, wherein the second material is located at one or both ends of the body.

8. The endoprosthesis of claim 7, wherein a cross-sectional portion between the ends of the body is free of the second material.

9. The endoprosthesis of claim 1, wherein the second material is located along a length of the body.

10. The endoprosthesis of claim 1, wherein the second material is located at a series of discontinuous portions along a length of the body.

11. The endoprosthesis of claim 1, wherein the second material extends spirally along the body.

12. The endoprosthesis of claim 1, wherein at least a portion of the second material is at least about five microns thick.

13. The endoprosthesis of claim 1, wherein the second material has a density greater than about 9.9 g/cm³.

14. The endoprosthesis of claim 1, wherein the second material is formed as two separate portions, each portion on opposing circumferential areas of the body.

15. The endoprosthesis of claim 1, wherein the second material is selected from the group consisting of tantalum, titanium, zirconium, iridium, palladium, hafnium, tungsten, gold, ruthenium, rhenium, barium, dysprosium, gadolinium and platinum.

16. The endoprosthesis of claim 13, wherein the second material includes an alloy.

17. The endoprosthesis of claim 1, further comprising a drug.

18. The endoprosthesis of claim 17, wherein the second material is disposed outwardly relative to the body.

19. The endoprosthesis of claim 1, further comprising a biodegradable coating on the body, the biodegradable coating comprising a third material having a third mass attenuation coefficient higher than the first mass attenuation coefficient.

20. A method, comprising:
obtaining an image of an endoprosthesis in a body using computed tomography, the endoprosthesis comprising a tubular body including a first material having a first mass attenuation coefficient, and a second material on less than or equal to half of a circumferential cross section defined by the body, the second material having a second mass attenuation coefficient greater than the first mass attenuation coefficient.

21. The method of claim 20, wherein the second material is on less than or equal to half of any circumferential cross section occupied by the body.

22. The method of claim 20, wherein the second material is on less than or equal to about forty percent of a circumferential cross section defined by the body.

23. The method of claim 20, wherein the second material is on less than or equal to about twenty percent of a circumferential cross section defined by the body.

24. The method of claim 20, wherein the second material is located at one or both ends of the endoprosthesis.

25. The method of claim 20, wherein a portion between ends of the endoprosthesis is free of the second material.

26. The method of claim 20, wherein the second material is disposed outwardly relative to the body.

27. The method of claim 20, wherein the second material is in a coating comprising a biodegradable material.

28. The method of claim 20, wherein the endoprosthesis further comprises a drug.

29. The method of claim 20, wherein obtaining the image includes determining a first and a second set of images from a plurality of computed tomography scan images, wherein the first set of images display a higher percentage of the second material than the second set of images.

30. The method of claim 29, further comprising forming a final image from the second set of images.

31. The method of claim 29, wherein determining from a plurality of computed tomography scan images a second set of images determines a set of images that display less than a predetermined amount of the second material.

32. A method for imaging an endoprosthesis, comprising:
obtaining a plurality of computed tomography scan images of a body having the endoprosthesis located therein;
determining from the plurality of computed tomography scan images, images that display the endoprosthesis;
subtracting selected images that display the endoprosthesis from the plurality of computed tomography scans to determine a set of desired images; and
forming a final image from the desired images.

33. The method of claim 29, wherein the endoprosthesis comprises a tubular body including a first material having a first mass attenuation coefficient, and a coating on less than or equal to half of any circumferential cross section defined by the body, the coating including a second material having a second mass attenuation coefficient greater than the first mass attenuation coefficient.

34. The method of claim 30, wherein the selected images display a higher percentage of the coating than a second set of images.
